(19) United States
(12) Patent Application Publication
(10) Pub. No.: US 2004/0138278 A1
(43) Pub. Date: Jul. 15, 2004

(54) Use of AT1 receptor antagonists for prevention of subsequent strokes

(75) Inventors: Harmut Kueppers, Hannover (DE); Jochen Schrader, Cloppenburg (DE)

Correspondence Address: CROWELL & MORING LLP, INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 10/612,391

(22) Filed: Jul. 3, 2003

(30) Foreign Application Priority Data
Jul. 5, 2002 (DE) 102 30 272.3

(51) Int. Cl. A61K 31/4184

(52) U.S. Cl. 514/381

(57) ABSTRACT

A method of preventing or inhibiting subsequent strokes by administering an AT₁-receptor antagonist, in particular eprosartan, to a patient who has previously suffered a first stroke.

### BACKGROUND OF THE INVENTION

The present invention relates to the use of AT₁-receptor antagonists for prevention of subsequent strokes.

Each year approximately five million people worldwide die of a stroke. Each year approximately fifteen million people have a non-fatal stroke, approximately one third of them sustaining substantial permanent health damage. Strokes are thus the third leading cause of death but the main cause of permanent health damage in adults.

Of the survivors of an acute (=first) stroke, approximately 20% have a subsequent or secondary stroke within the next five years. A stroke in the sense of the present invention is understood to refer to a cerebrovascular event, such as a transient ischemic attack, cerebral ischemia and intracerebral hemorrhaging.

It is known that high blood pressure is one of the most important risk factors for an acute (first) stroke. Consequently, the risk of hypertensive patients suffering an acute stroke can be reduced statistically significantly through antihypertensive therapy. Antihypertensive therapy for the purpose of preventing acute strokes can be administered with comparable results by using compounds such as those from the pharmacological classes of beta-blockers, calcium antagonists, diuretics or in some cases even a combination therapy, including the use of compounds from the ACE inhibitor class. The suitability of the AT₁-receptor antagonist candesartan for antihypertensive therapy during the acute phase of a stroke is currently the subject of a clinical trial (ACCESS Study, see J. Schrader et al., Basic Res. Cardiol. 93, Suppl. 2 (1998), 69-78), for example. Results of this study are not yet available.

However, in prevention of subsequent (e.g., secondary) strokes, no clear correlation can be found between lowering elevated blood pressure and reducing a (male or female) patient's risk of suffering a subsequent stroke. A subsequent stroke is a stroke which occurs in the same person after at least one prior initial stroke. In particular, a secondary stroke should be understood to refer to a new stroke occurring after just one prior stroke in the same patient.

To investigate the circumstances that can influence the risk of a subsequent stroke, a long-term clinical study has been conducted on stroke patients. The hypertensive and nonhypertensive patients participating in this study received as the active ingredient the ACE inhibitor perindopril either alone (=ACE monotherapy) or in combination with the diuretic indapamide (=ACE combination therapy) (=PROGRESS Study, see, for example, The PROGRESS Collaboration Group, The Lancet 358 (2001), 1033-1041). In this clinical study, it was found that the blood pressure of patients in the hypertensive group could be lowered to a comparable extent by ACE monotherapy as well as ACE combination therapy. Nevertheless, the risk of suffering a subsequent stroke dropped significantly only in the subgroup of hypertensive patients treated with the ACE combination therapy, whereas for the subgroup of hypertensive patients treated with ACE monotherapy, the risk of suffering a subsequent stroke did not differ from the corresponding risk in the placebo patient group. Another finding of the PROGRESS study was that the reduction in risk, if any, of suffering a subsequent stroke was of approximately the same order of magnitude in both the hypertensive and nonhypertensive patient groups.

### SUMMARY OF THE INVENTION

Accordingly, it is an object of the invention to provide a method of preventing or inhibiting a secondary or subsequent stroke in a patient who has previously suffered an initial stroke.

This and other objects are achieved in accordance with the present invention by providing a method of preventing or inhibiting a subsequent stroke in a patient who has previously suffered at least one earlier stroke, said method comprising administering to such a patient an effective subsequent stroke inhibiting amount of an AT₁-receptor antagonist.

In a preferred embodiment, the AT₁-receptor antagonist is administered to prevent a secondary stroke in a patient who had previously experienced just one earlier stroke.

It has now surprisingly been found that by administering the AT₁-receptor antagonist as the only active ingredient (AT₁ monotherapy), a patient's risk of suffering a subsequent stroke can be reduced significantly. Therefore, the present invention relates to the use of AT₁-receptor antagonists for prevention of subsequent strokes.

AT₁-receptor antagonists are pharmacological active ingredients which can selectively block the AT₁ subtype of the angiotensin II receptor in large mammals, humans in particular, and are known as antihypertensive agents. Particularly suitable AT₁-receptor antagonists for use in the present invention include candesartan, eprosartan, irbesartan, losartan, telmisartan and/or valsartan and their physiologically acceptable acid addition salts. It should be understood, however, that other AT₁-receptor antagonists also may be used within the scope of the present invention. Eprosartan or a physiologically acceptable acid addition salt thereof is preferred. It is especially advantageous to use eprosartan mesylate, in particular eprosartan mesylate monohydrate.

Candesartan and its physiologically acceptable acid addition salt are known per se, e.g., from European Patent Application No. EP 459,136 (see also U.S. Pat. No. 5,196,444). Candesartan can be synthesized by the synthesis process described in the foregoing publication or by methods similar to these synthesis processes.

Eprosartan and its physiologically acceptable acid addition salts are known per se, e.g., from European Patent Application No. EP 403,159 (see also U.S. Pat. No. 5,185,351). Eprosartan can be synthesized by the synthesis processes described in the foregoing publication or by other synthesis processes, e.g., those known from the publications WO 98/35962 or WO 98/35963 or by other similar synthesis processes. Eprosartan mesylate, for example, is known from European Patent Application No. EP 403,159 and can be obtained by the synthesis processes described in that publication. Eprosartan mesylate monohydrate, for example, is known from WO 99/00383 and can be synthesized by the method described in this publication. WO 92/10188 discloses the use of eprosartan, among others, for treatment of hemorrhagic stroke.

Irbesartan and its physiologically acceptable acid addition salts are known per se, e.g., from European Patent Application No. EP 454,511 (see also U.S. Pat. No. 5,270,317). Irbesartan can be synthesized by the synthesis processes described in the foregoing publication or by methods similar to those.

Losartan and its physiologically acceptable acid addition salts are known per se, e.g., from European Patent Application No. EP 253,310 (see also U.S. Pat. No. 5,138,069). Losartan can also be synthesized by the synthesis processes described in the foregoing publication or by similar procedures.

Telmisartan and its physiologically acceptable acid addition salts are known per se, e.g., from European Patent Application No. EP 502,314. Telmisartan can also be synthesized by the synthesis processes described in the foregoing publication or by similar procedures.

Valsartan and its physiologically acceptable acid addition salts are known per se, e.g., from European Patent Application No. 443,983 (see also U.S. Pat. No. 5,399,578). Valsartan can also be synthesized by the synthesis processes described in the publication cited above or by similar procedures.

Prevention is understood to refer to preventive care for patients to prevent or at least inhibit subsequent strokes, in particular preventive care to inhibit a subsequent stroke after just one prior acute (first) cerebrovascular event. Prevention of a subsequent stroke after just one prior initial stroke is usually referred to as secondary stroke prevention. Use of AT₁-receptor antagonists according to this invention for secondary stroke prevention is preferred. Usually a patient requiring subsequent stroke prevention will take an amount of at least one AT₁-receptor antagonist adequate to reduce his risk of suffering a subsequent stroke and will take it for a lengthy period of time, under some circumstances permanently, as maintenance therapy.

### DESCRIPTION OF THE CLINICAL EXPERIMENTAL METHOD

A clinical study is currently being conducted on 1369 patients, investigating the suitability of antihypertensive agents of various pharmacological classes to reduce a patient's risk of suffering a subsequent stroke.

Of the 1369 patients (male/female), 691 patients received the AT₁-receptor antagonist eprosartan (as eprosartan mesylate); 678 of the 1369 patients received the calcium antagonist nitrendipine.

The study is being conducted as a prospective, randomized, controlled and multicentric study and corresponds to the PROBE design (=prospective, randomized, open-blinded endpoint). The primary target criterion is the evaluation of the total mortality and the total number of cardiovascular and cerebrovascular events (strokes) in the sense of this invention.

The subsequent efficacy parameter is the change in mental abilities (evaluated on the basis of neurological findings, Barthel index and Rankin scale). In addition, the change in the average blood pressure over a period of time is evaluated in measurements performed on the patients sitting down in the office as well as in ambulatory 24-hour blood pressure measurements.

The study begins within 24 months after the occurrence of cerebral ischemia or an intracerebral hemorrhage in a patient. The patients are observed for up to four years. The essential prerequisite for including a patient in the study is hypertension requiring treatment and the condition following a cerebral ischemia, a transitory ischemic attack or an intracerebral hemorrhage within the last 24 months. Essential exclusion criteria include an occlusion or stenosis of greater than 70% of the internal carotid artery (ICA), manifest cardiac insufficiency (NYHY [sic; NYHA] class III-IV), age of patient greater than 85 years at the time of the cerebrovascular event, anticoagulant therapy for a patient because of cardiac arrhythmia, high-grade aortic or mitral valve stenosis, unstable angina pectoris or a known hypersensitivity to AT₁-receptor antagonists or calcium antagonists of the dihyropyridine type or chemically related substances.

After inclusion in the study, the patients begin the randomized treatment phase with eprosartan or nitrendipine. Depending on the blood pressure readings and patient-specific criteria, treatment begins with 600 mg eprosartan once daily or 10 mg nitrendipine q.d. For long-term therapy, a diastolic blood pressure of less than 90 mmHg sitting down and a systolic blood pressure of less than 140 mmHg are the goals. For the 24-hour blood pressure measurements, a daily average of less than or equal to 135/85 mmHg is the goal. If additional antihypertensive therapy is necessary, conventional recognized criteria such as those recommended by the German Hypertension League are followed in the combination therapy. Combination therapy with ACE inhibitors, AT₁-receptor antagonists or calcium antagonists should not be used.

Study end points (target criteria) are transmitted immediately to the central study office, where they are treated as open-blind results with respect to the medication and then forwarded to the End Point Committee. The committee performs the evaluation and classification of the findings with regard to a cardiovascular or cerebrovascular end point.

In an interim report on this study, the data on a total of 337 of the participating patients has been evaluated; 179 of these 337 patients received eprosartan (eprosartan group) and 158 of these 337 patients received nitrendipine (nitrendipine group).

The average age of the patients in the eprosartan group was 70.2 years; 93 patients were male and 86 patients were female. The average observation period for a patient at the time of monitoring was 18.4 months. The patients were included in the study an average of 29.5 months after their stroke.

In the nitrendipine group, the average age of the patients was 70.1 years; 76 patients were male and 82 patients were female. The average observation period for a patient at the time of monitoring was 18.3 months. The patients were included in the study an average of 29.8 months after their stroke.

As an intermediate finding, 15 subsequent cerebrovascular events in the sense of this invention had occurred in the eprosartan group (=8.4%) by the time of the monitoring, versus 22 such subsequent cerebrovascular events in the nitrendipine group (=13.9%).

In comparison, the incidence of subsequent cerebrovascular events in the PROGRESS study according to the protocol was 13.7% (420/3054 patients) in the placebo group. The incidence of cerebrovascular events in the patient group of the PROGRESS study treated with the active ingredient (according to the definition of the PROGRESS study) was 10.6% (307/3051 patients).

It can be seen from the clinical research findings presented above that a significant reduction in a patient's risk of having a subsequent stroke can be achieved through AT₁ monotherapy in particular monotherapy with eprosartan. This success achieved with AT₁ monotherapy in the inhibition of subsequent strokes is much better than the success achieved with methods investigated so far and is much better than the corresponding results obtained with a placebo group of patients at risk of a cerebrovascular event.

It can thus be concluded that AT₁-receptor antagonists, preferably eprosartan, yield an especially pronounced reduction in a patient's risk of suffering a subsequent stroke and that AT₁-receptor antagonists are especially effective in preventing or inhibiting subsequent strokes, in particular for secondary stroke inhibition.

The doses of the AT₁-receptor antagonists used may vary individually and will of course also vary according to the type of substance used and the form of administration. In general, however, the pharmaceutical dosage forms known from antihypertensive therapy for AT₁-receptor antagonists and having an active ingredient content of 10 mg to 700 mg, in particular 50 mg to 600 mg, per single dose are suitable for administration to large mammals, in particular humans.

As medicinal agents, the AT₁-receptor antagonists can be formulated with conventional pharmaceutical excipients in galenical preparations such as tablets, capsules, suppositories or solutions. These galenical preparations can be produced according to known methods using conventional solid or liquid vehicles such as lactose, starch or talc or liquid paraffins and/or using conventional pharmaceutical excipients, e.g., tablet disintegrants, solubilizers or preservatives. The production of galenical preparations of AT₁-receptor antagonists is known per se, e.g., from the publications cited above as sources of the production processes for the particularly suitable AT₁-receptor antagonists according to this invention.

The pharmaceutical preparations according to this invention can be produced by methods similar to those already known. For example, pharmaceutical preparations according to this invention, in particular preparations containing eprosartan, can be produced by methods like those described in such publications as European Patent Application No. EP 403,159 and International Patent Application No. WO 99/45779.

The following example is presented to describe the present invention in greater detail without restricting its scope in any way.

### EXAMPLE I

### Capsules Containing Eprosartan Mesylate

Capsules are prepared with the following composition per capsule:

| | |
|---|---|
| (E)-α-[[2-butyl-1-[(4-carboxyphenyl)methyl]-1H-imidazol-5-yl]- | 50 mg |
| methylene]-2-thiophenepropanoic acid monomethanesulfonate | |
| (= eprosartan mesylate) | |
| Cornstarch | 60 mg |
| Lactose | 270 mg |
| Ethyl acetate | as |
| | needed |

The active ingredient, cornstarch and lactose are mixed thoroughly with the help of ethyl acetate and processed to form a homogenous, pasty mixture. The paste is pulverized, and the resulting granules are applied to a suitable metal plate and dried at 45° C. to remove the solvent. The dried granules are passed through a pulverizing machine and mixed with the following additional excipients in a mixer:

| | |
|---|---|
| Talc | 5 mg |
| Magnesium stearate | 5 mg |
| Cornstarch | 9 mg |

and then filled into 400 mg capsules (=capsule size 0).

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method of preventing or inhibiting a subsequent stroke in a patient who has previously suffered at least one earlier stroke, said method comprising administering to such a patient an effective subsequent stroke inhibiting amount of an AT₁-receptor antagonist.

2. A method according to claim 1, for inhibiting a secondary stroke, wherein said patient has experienced only one earlier stroke.

3. A method according to claim 1, wherein the AT₁-receptor antagonist is selected from the group consisting of candesartan, eprosartan, irbesartan, losartan, telmisartan, valsartan and physiologically acceptable acid addition salts thereof.

4. A method according to claim 3, wherein the AT₁-receptor antagonist is eprosartan or a physiologically acceptable acid addition salt thereof.

5. A method according to claim 4, wherein the AT₁-receptor antagonist is eprosartan mesylate.
